(19) United States
(12) Patent Application Publication
(10) Pub. No.: US 2020/0001107 A1
(43) Pub. Date: Jan. 2, 2020

(54) LIGHT TREATMENT SYSTEM

(71) Applicant: Johnson & Johnson Consumer Inc., Skillman, NJ (US)

(72) Inventor: Curt Binner, Skillman, NJ (US)

(21) Appl. No.: 16/455,154

(22) Filed: Jun. 27, 2019

Related U.S. Application Data
(60) Provisional application No. 62/691,718, filed on Jun. 29, 2018.

(51) Int. Cl. A61N 5/06

(52) U.S. Cl. CPC A61N 5/0616; A61N 2005/067; A61N 5/062

(57) ABSTRACT

A phototherapy system wearable on a portion of a user's face includes a lamp platform and a controller unit disposed on, and electrically coupled to, the lamp platform, and a frame for holding the lamp platform in a fixed orientation on a user's face. The controller unit has an inwardly facing surface directed toward the user's face during use; a rechargeable power source; and a charging receptacle disposed in the inwardly facing surface and in electrical communication with the charging receptacle. In use, the frame holds the lamp platform and controller unit in a relationship such that the spacing between the charging receptacle and a closest surface of the user's face defines a charging receptacle clearance.

### FIELD OF THE INVENTION

The present invention relates to devices and methods for delivering light-based skin therapy treatments for improving skin health. Specifically, the invention is a phototherapy system wearable on a portion of a user's face for improving skin health.

### BACKGROUND OF THE INVENTION

Light therapy, also known as phototherapy, or heliotherapy, consists of exposure to daylight or to specific wavelengths of light using polychromatic polarized light, lasers, light-emitting diodes (LEDs), fluorescent light, dichroic light or very bright, full-spectrum light. The light is administered for a prescribed amount of time and, in some cases, at a specific time of day.

Skin disorders treated with light therapy include: atopic dermatitis, psoriasis, vitiligo, acne vulgaris, eczema, neonatal jaundice, and some forms of cancer.

There are many known devices for the administration of light therapy to patients. The size of device needed depends on size of the area that needs treatment. Skin disorders can involve just a few small patches, to nearly the entire body. So, devices for use in skin treatment include floor, countertop or hand-help lamps, as well as wearable patches and masks.

Consumers can visit a doctor's office to receive treatments, but convenient at-home light therapy delivery devices are also desired. Many of these devices need to be hand-held, which generally have not proven satisfactory. A hands-free therapeutic experience is always better than having to hold the device in a particular position for extended periods of time during the therapy.

Recently, a number of phototherapy treatment devices for treating a user's face have been introduced. Many of these, in the form of masks, are flexible to conform to different sizes and shapes, and are simple to use without user discomfort. The phototherapy treatment devices for treating a user's face come in the form of an assembly which includes a wearable therapeutic lamp platform including a plurality of radiant lamps emitting radiant energy, a frame supporting the therapeutic lamp platform on the user and positioning the therapeutic lamp platform on the user, and a controller operatively associated with the therapeutic lamp platform.

Often the controller is separated from, and tethered to, the therapeutic lamp platform. This makes use of the phototherapy treatment device less convenient, as the user must hold the controller in one hand while wearing the therapeutic lamp platform on their face.

To overcome this issue, the controller can be mounted on the therapeutic lamp platform. However, the size/weight of the controller, as well as the difficulty of placing the controller on the mask at a location that does not distract from the user's comfort when the phototherapy treatment device is in use.

Also, many of the phototherapy treatment devices for treating a user's face available on the market come with controllers with rechargeable power sources. In recent years, there have been numerous reports world-wide of rechargeable batteries, specifically lithium batteries, overheating during recharging, and causing damaging fires. These were due to either battery failure, or the user charging the battery with the wrong kind of battery charger. It is therefore not recommended to recharge the batteries of the phototherapy treatment device while wearing the device.

It is desired to provide means of using the benefits of the phototherapy in a manner to maximize therapeutic efficiencies in exposure while maintaining ease, convenience, and safety of use. For this reason, light weight, flexible, adjustable phototherapy treatment devices for treating a user's face are disclosed, incorporating controllers with rechargeable power sources mounted on the therapeutic lamp platform, which, for user safety, cannot be recharged while the user is wearing the device.

### SUMMARY OF THE INVENTION

In one embodiment, the invention relates to a phototherapy system wearable on a portion of a user's face, the system comprising:
(a) a lamp platform having an outer surface and an opposite, inner surface directed toward the user's face during use and comprising a plurality of treatment lamps arranged and configured to irradiate a portion of the user's face;
(b) a controller unit disposed on, and electrically coupled to, the lamp platform, the controller unit having:
   (i) an inwardly facing surface directed toward the user's face during use;
   (ii) a rechargeable power source; and
   (iii) a charging receptacle disposed in the inwardly facing surface and in electrical communication with the charging receptacle;
(c) a frame for holding the lamp platform in a fixed orientation such that the inner surface of the lamp platform and the inwardly facing surface of the controller unit are spaced from the user's face and the spacing between the inner surface of the lamp platform and an adjacent surface of the user's face defines a platform clearance and the spacing between the charging receptacle and a closest surface of the user's face defines a charging receptacle clearance.

In another embodiment, it relates to a phototherapy system wearable on a portion of a user's face, the system comprising:
(a) a lamp platform having
   (i) an outer surface and an opposite, inner surface directed toward the user's face during use and comprising a plurality of treatment lamps arranged and configured to irradiate a portion of the user's face;
   (ii) the lamp platform has a height (h) extending from a base to a top that defines a longitudinal axis, and the lamp platform has a generally concave form in a lateral plane (perpendicular to the longitudinal axis);
(b) a controller unit disposed on and electrically coupled to the base of the lamp platform, the controller unit having an inwardly facing surface directed toward the user's face during use and a rechargeable power source, and the inwardly facing surface of the controller unit has a generally concave form in a lateral plane (perpendicular to the longitudinal axis); and
(c) a frame for holding the lamp platform in a fixed orientation such that the inner surface of the lamp platform is spaced from the user's face, and the spacing between the inner surface of the lamp platform and an adjacent surface of the user's face defines a platform clearance.

In a third embodiment it relates to a method of treatment using the phototherapy system of claim 1 comprising inserting a cable connector into the charging receptacle for a time sufficient to energize the rechargeable power source, removing the cable connector from the charging receptacle to enable the user to place the phototherapy system on the face, initiating a program of phototherapy managed by the controller unit that irradiates at least a portion of the user's face with one or more of the treatment lamps, allowing the program of phototherapy to complete the phototherapy, and removing the phototherapy system from the face.

### BRIEF DESCRIPTION OF THE DRAWINGS

FIG. 1 is a front perspective view of an embodiment of a phototherapy system of the present invention;

FIG. 2 is a front view of the system of FIG. 1;

FIG. 3 is a rear view of the system of FIG. 1;

FIG. 4 is an exploded perspective view of the system of FIG. 1;

FIG. 5 is a front view of a controller unit disposed on the wearable lamp platform of the phototherapy system;

FIG. 6 is a rear view of the controller unit of FIG. 5;

FIG. 7 is an exploded perspective view of the controller unit of FIG. 5;

FIG. 8 is a view of the second side of the front panel of the controller unit of FIG. 5;

FIG. 9 is a view of the second side of the front panel of the controller unit of FIG. 5 disposed on the wearable lamp platform;

FIG. 10 is a view of the second side of the rear panel of the controller unit of FIG. 5;

FIG. 11 is a top view of a partially assembled controller unit of FIG. 5, with the controller unit disposed in the front panel of the controller unit;

FIG. 12 is a rear view of a partially assembled controller unit of FIG. 5, with the controller unit disposed in the front panel of the controller unit;

FIG. 13 is a rear perspective view of the phototherapy system of FIG. 1 coupled to a charging cable; and

FIG. 14 is a side perspective view of the phototherapy system of FIG. 1 coupled to a charging cable.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs.

The present invention relates to devices and methods for delivering light-based skin therapy treatments for improving skin health. Specifically, the invention is a phototherapy system wearable on a portion of a user's face for improving skin health. Therapy treatments include, but are not limited to, anti-aging enhancement or acne prevention. The device is wearable and hands-free. The phototherapy system 10 disclosed herein has a controller unit disposed on, and electrically coupled to, the lamp platform, with the safety feature of preventing the charging of system 10 while the user is wearing the device.

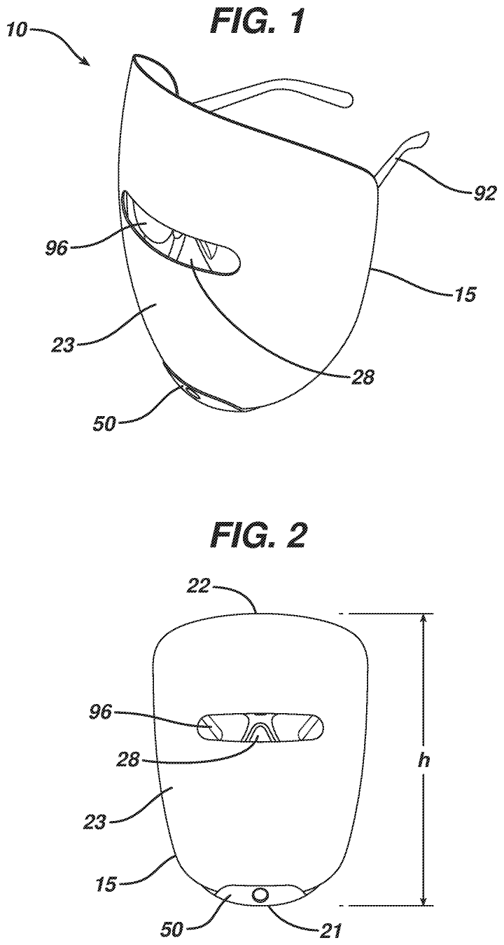

FIGS. 1-4 show an embodiment of a phototherapy system 10 wearable on a portion of a user's face. Phototherapy system 10 includes a wearable lamp platform 15, a controller unit 50 disposed on, and electrically coupled to, lamp platform 15, and a frame 90 for holding wearable lamp platform 15 in a fixed orientation spaced from the user's face.

Wearable lamp platform 15 has a base 21, a top 22, an outer surface 23, an opposite inner surface 34 directed toward the user's face during use, a plurality of treatment lamps 82 arranged and configured to irradiate a portion of a user's face, and an eye slot 28 extending through lamp platform 15 from outer surface 23 to inner surface 34.

Inner surface 34 of wearable lamp platform 15 is reflective and is arranged and configured to reflect light scattered by the user's face back to the face.

Wearable lamp platform 15 has a height (h) extending from base 21 to top 22, that defines a longitudinal axis, and lamp platform 15 has a generally concave form in a lateral plane (perpendicular to the longitudinal axis).

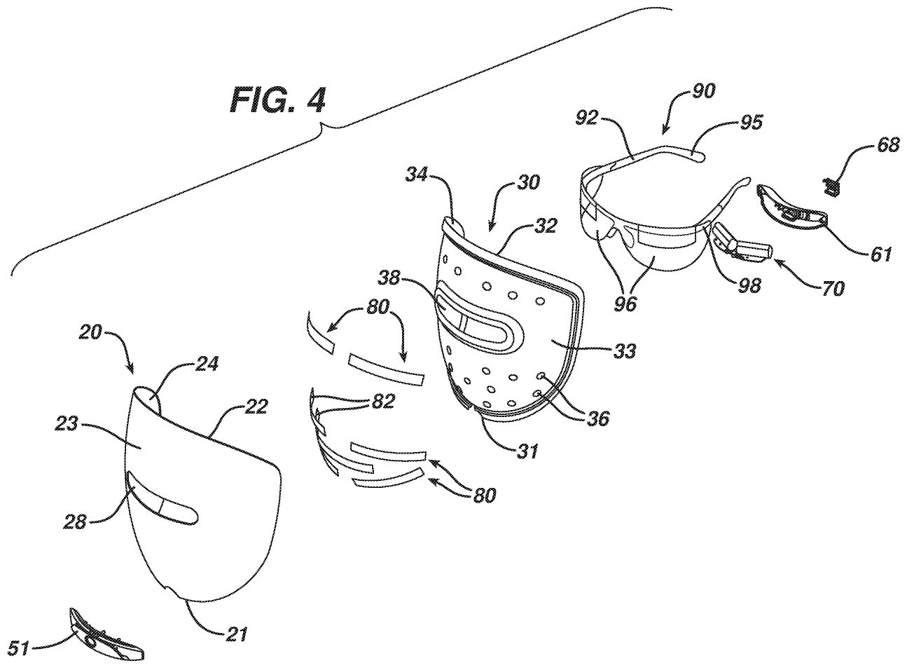

FIG. 4 is an exploded perspective view of the phototherapy system. As seen in the figure, wearable lamp platform 15 is comprised of an outer wall 20 and an inner wall 30. Outer wall 20 is disposed furthest away from user's face during treatment, while the inner wall 30 is disposed closer thereto. The walls have a concave configuration in both horizontal and vertical directions, where the concavity comprises a multi-dimensional parabolic curvature for catching and reflecting the radiation back to the treatment areas. In this embodiment, the walls are constructed of a plastic material having a malleable rigidity so that wearable lamp platform 15 can be bent and deflected slightly during use. It is intended that the concavity is slightly smaller than the head of the user so that the mask is bent out when applied thereby providing a close but comfortable tightness on the user which will keep the wearable lamp platform 15 in a desired position during use.

Outer wall 20 has a base 21, a top 22, an outer wall first surface 23 (also known as outer surface), an outer wall second surface 24, and an eye slot 28 extending through outer wall 20 from outer wall first surface 23 to outer wall second surface 24.

Inner wall 30 has a base 31, a top 32, an inner wall first surface 33, an inner wall second surface 34 (also known as inner surface), snap-out pivotal connections 35, and treatment lamp apertures 36 and an eye slot 38 extending through inner wall 30 from inner wall first surface 33 to inner surface 34. Inner surface 34 is comprised of a smooth seamless reflective surface facing the treatment area.

Outer wall 20 and inner wall 30 have different radii of concavity. When wearable lamp platform 15 is assembled, the entire perimeter is sealed as outer wall 20 and inner wall 30 come together. Such a mating seal is typically effected through a sonic weld arrangement. Alternatively, local sealing points (not shown) can be employed to assemble the walls together with spaced intermediate seals. As far as the user is concerned wearable lamp platform 15 presents an integral structure.

When wearable lamp platform 15 is assembled, treatment lamp apertures 36 are matingly aligned relative to treatment lamps 82 so that lamps 82 can radiate the therapeutic light through apertures 36. Accordingly, treatment lamps 82 are recessed relative to inner wall 30 to preclude contact with the treatment surface and to make it very difficult for treatment lamps 82 themselves to be in any way contacted by the user. Such an assembly results in a controlled communication of radiating therapy in a manner to impart a predetermined cone of therapeutic light on to a treatment area. Treatment lamp apertures 36 are disposed relative to desired treatment areas and wall parabolic configuration for even light distributions across the treatment area. A combination of such a controlled cone of light, predetermined disposition of treatment lamps 82 themselves on wearable lamp platform 15, an inner reflective surface on inner surface 34 of inner wall 30, and a controlled positioning of the assembly relative to the treatment area via a platform position relative to contact areas of the nose and the ears, presents an assembly which presents a highly predictable distributive pattern of the light (predetermined cones of light per light source), thereby minimizing the number of treatment lamps 82 that need to be included for effective treatment.

In addition, when assembled, there is a spacing between outer wall 20 and inner wall 30 of wearable lamp platform 15. Disposed in the spacing, for enhanced safety and convenience purposes, are treatment lamps 82 and the circuitry connecting the lamps to power source controller 50. As shown in FIG. 4, treatment lamps 82 are disposed on treatment lamp platforms 80. Snap-out pivotal connection bodies 35 are also disposed on inner surface 34 of inner wall 30. Though not shown in the drawings, circuitry connects power source controller 50 to treatment lamps 82. The circuitry may be in the form of conductive wires or filaments. They may be made of metallic or nonmetallic conducting materials. Metallic conducting materials include copper, aluminum and silver. Nonmetallic conducting materials include graphite or conductive polymers.

In some embodiments, outer wall 20 primarily functions as a support for treatment lamps 82, lenses and the circuitry. Alternatively, treatment lamps 82 could be fixed to the inner wall 30. Regardless of which wall supports treatment lamps 82, the lamps 82 need to be properly aligned with treatment lamp apertures 36 to achieve desired performance of wearable phototherapy system 10.

While the inner wall 30 is described above having treatment lamp apertures 36 aligned with treatment lamps 82, one of ordinary skill will recognize that other light transmissive schemes may be used, such as light transmissive windows in the inner wall and even a continuously transmissive inner wall, such as a clear plastic inner wall.

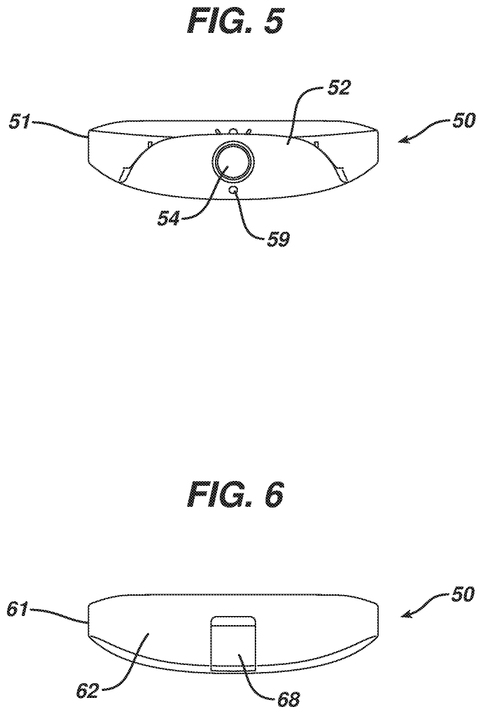

Controller unit 50, shown in FIGS. 5 through 12, is disposed on, and electrically coupled to, wearable lamp platform 15. The unit is attached to base 21 of outer wall 20 as well as base 31 of inner wall 30. The components of controller unit 50 include a front piece 51, a rear piece 61, and an electronics unit 70.

FIG. 5 is a front view of controller unit 50. The figure shows front panel 51 with first side 52 of front panel 51, a power button flap 54, and an indicator light 59. FIG. 6 is a rear view of the controller unit 50. The figure shows rear panel 61 with an inwardly facing surface 62 of controller unit 50, and a charging aperture cover flap 68. Inwardly facing surface 62 on rear panel 61 of controller unit 50 is directed toward the user's face during use of wearable phototherapy system 10.

FIG. 7 is an exploded perspective view of the controller unit 50. The figure shows front panel 51, rear panel 61, and electronics unit 70.

Electronics unit 70 has a controller comprised of an Printed Circuit Board Assembly (“PCBA”) 72, a rechargeable power source 74 in the form of two elongate, cylindrical cells, and a charging receptacle 76. The two elongate, cylindrical cells may be, for example, rechargeable batteries.

As mentioned earlier, wearable lamp platform 15 has a height (h) extending from base 21 to top 22, that defines a longitudinal axis, and lamp platform 15 has a generally concave form in a lateral plane (perpendicular to the longitudinal axis). FIG. 7 shows the two elongate, cylindrical cells comprising rechargeable power source 74 in defining an oblique angle therebetween in the lateral plane. The oblique angle of the two elongate, cylindrical cells generally followed the concave form at the base 21 of lamp platform 15. This prevents electronics unit 70 from jutting out from inner wall 30 in the direction of the user's face.

Rear panel 61 of controller unit 50, as shown in FIG. 6, has inwardly facing surface 62, and a charging aperture cover flap 68. During use of wearable phototherapy system 10, inwardly facing surface 62 is directed toward the user's face. Charging receptacle 76, not shown in FIG. 6, is disposed in controller unit 50, and as shown in the figure, covered by charging aperture cover flap 68.

Though not limiting, charging receptacle 76 may be in the form of a USB receptacle. USB, short for Universal Serial Bus, is an industry standard that was developed to define cables, connectors and protocols for connection, communication, and power supply between personal computers and their peripheral devices. Generally, USB receptacles are available as standard USB Type-A, Type-B, or Type-C. Recent developments have also included “mini” and “micro” USB receptacles, such as, but not limited to, Mini-A, Mini-AB, Mini-B, Micro-A, Micro-AB, and Micro-B. In the embodiment shown, charging receptacle 76 is a USB Micro-A.

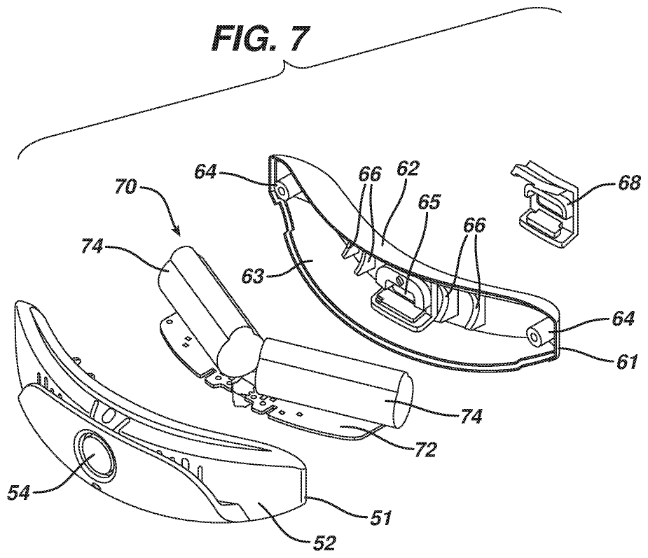

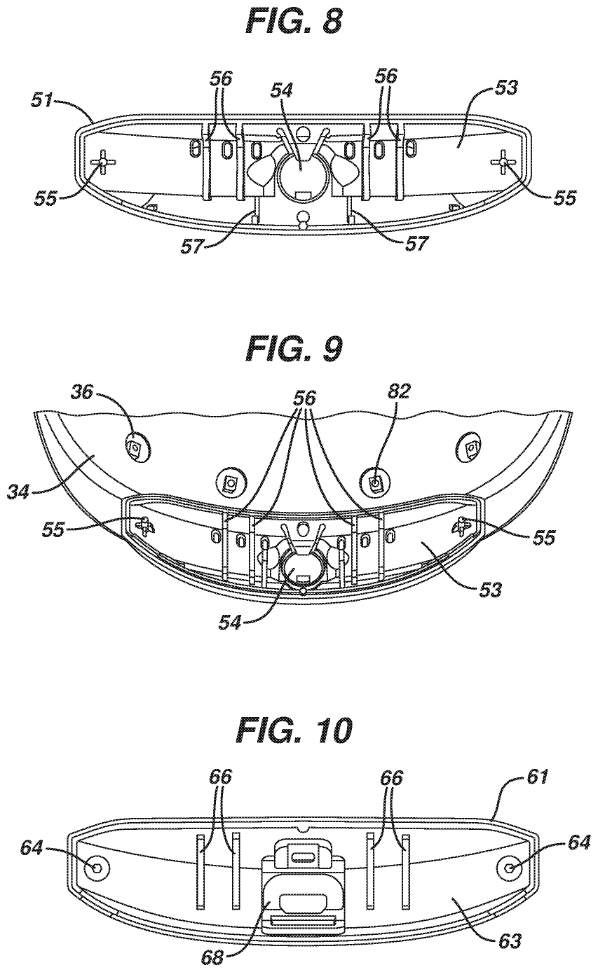

FIG. 8 is a view of the second side 53 of front panel 51 of controller unit 50. Second side 53 of front panel 51 includes power button flap 54, the male portion 55 of a snap fastener (or snap) used to attach front panel 51 to rear panel 61 of controller unit 50, and power source clips 56 and PCBA clips 57. Male portion 55 of the snap fastener is disposed in the female portion 64 of the snap fastener when controller unit 50 is assembled. Although snap fasteners are used to assemble controller unit 50, other means of attaching front panel 51 to rear panel 61 are also contemplated. These include screws, pins, hooks, or adhesives. In some embodiments, combinations of snap fasteners or screws with adhesives may be used to insure solid construction of controller unit 50.

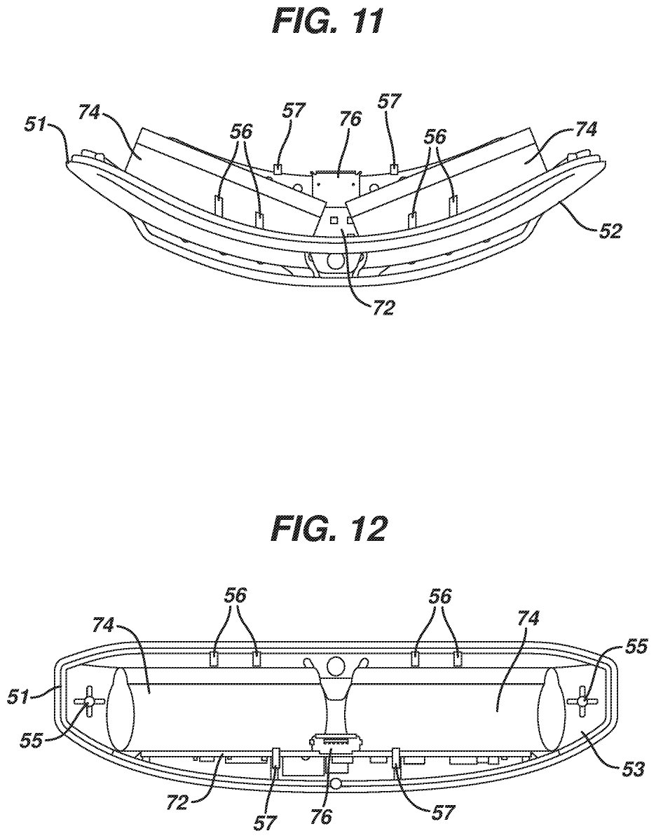

Power source clips 56 and PCBA clips 57 are used to hold rechargeable power source 74 (such as rechargeable batteries) and PCBA 72 in place when controller unit 50 is assembled. FIGS. 11 and 12 show clips 56 and 57 with electronics unit 70 disposed within.

FIG. 9 is a rear view of front panel 51 the controller unit 50 disposed on wearable lamp platform 15. As mentioned above, unit 50 is disposed on and attached to base 22 of outer wall 20 as well as base 32 of inner wall 30. In this embodiment, base 22 and base 32 are notched for placement of controller unit 50 on wearable lamp platform 15. In some embodiments, controller unit 50 is attached to wearable lamp platform 15 by means of snaps, screws, pins, hooks, or adhesives. In some embodiments, combinations of snap fasteners or screws with adhesives may be used. In the embodiment presented, an adhesive is used to attach controller unit 50 to wearable lamp platform 15.

As mentioned above, rear panel 61 has an inwardly facing surface 62, also known as the inwardly facing surface of controller unit 50, or the first side or rear panel 61. During use of wearable phototherapy system 10, inwardly facing surface 62 is directed toward the user's face. Second side 63 of the rear panel 61 of the controller unit 50 is shown in FIG. 10. Second side 63 of the rear panel 61 includes female portion 64 of a snap fastener, charging aperture 65, power source clips 66 and charging aperture cover flap 68. As mentioned above, male portion 55 of the snap fastener is disposed in the female portion 64 of a snap fastener when controller unit 50 is assembled. Power source clips 66 and are used to hold rechargeable power source 74 (such as rechargeable batteries) in place when controller unit 50 is assembled.

Charging receptacle 76 is disposed in the inwardly facing surface 62 of controller unit 50, and is in electrical communication with the rechargeable power source 74. Charging aperture 65 needs to be properly aligned with charging receptacle 76 to achieve desired charging function. Charging aperture cover flap 68 is hinged so that it may pivot to a position to allow a connection to rechargeable power source 74.

Frame 90, shown in the exploded view of FIG. 4, is a frame used for holding wearable lamp platform 15 in a fixed orientation spaced from the user's face. In addition, frame 90 is used for holding lamp platform 15 in a fixed orientation such that the inner surface of lamp platform 15 and the inwardly facing surface 62 of controller unit 50 are spaced from the user's face and the spacing between the inner surface 34 of inner wall 30 of the lamp platform 15 and an adjacent surface of the user's face defines a platform clearance and the spacing between charging receptacle 76 and a closest surface of the user's face defines a charging receptacle clearance.

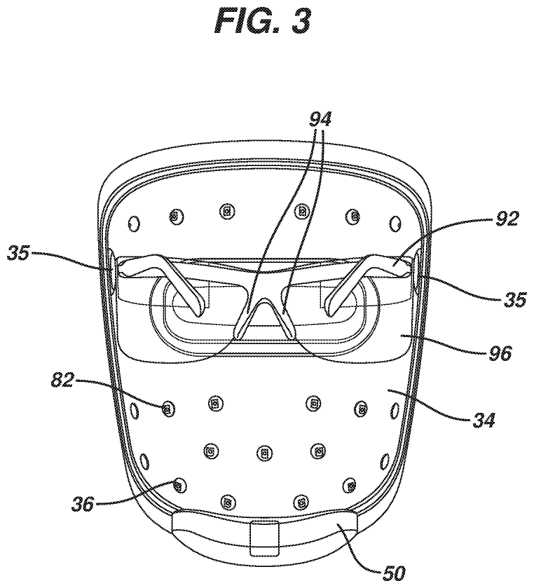

Frame 90, as shown in FIGS. 3 and 4, has temple arms 92, nose arms 94, ear latches 95, lenses 96, and connectors 98. Temple arms 92 are the long arms on the sides of frame 90 that extend over the ears. Ear latches 95 wrap partially around the user's ears, and keep frame 90 on the user's face, especially if the user tilts their head down. Nose arms 94 hold wearable lamp platform 15 in a set distance from the user's face. Lenses 96 provide protection to the user's eye from treatment lamps 82. In some embodiments, interchangeable lenses 96 can be used to optimize user comfort. Connectors 98 attach frame 90 to snap-out pivotal connections 35 on inner wall 30 of lamp platform 15. In some embodiments, temple arms 92 may telescope for better sizing relative to the head size of the user, or could include a head strap to secure wearable lamp platform 15 to the user.

Snap-out pivotal connections 35 allow wearable lamp platform 15 to pivot relative to frame 90 so that a user may adjust light intensity relative to a treatment area by moving the platform closer or farther away. As noted above, platform 15 is flexible with a concave parabolic bias, but still has a malleable rigidity. When frame 90 is received on the user, it is disposed to expand lamp platform 15 parabolic bias to form a match to the size of the user's head. Frame 90 reference contact points to the user may comprise the temples, the nose bridge and the ears of the user.

Treatment lamps 82 may be Light Emitting Diodes (LEDs), or other radiant energy forms. This includes fluorescents, lasers, infrareds, ultraviolet or combinations of radiant energy forms. Methods of manipulating the light energy are encompassed within the present embodiments. Other methods of light emission may comprise continuous, pulsed, focused, diffuse, multi-wavelength, single wavelength, visible and/or non-visible light wavelengths.

Treatment lamps may provide blue light having a peak wavelength of between about 450 nanometer (nm) and about 495 nm, or red light having a peak wavelength of between about 620 nm and about 700 nm, or infrared light having a peak wavelength of between about 700 nm and about 1000 nm.

The wearable lamp platform 15 embodiment shown has a total of twenty-one treatment lamps 82 arranged in an orderly pattern to cover the jaw line, chin, cheek, nose, and forehead, but not the eyelids of the user. The number, arrangement, type, and color of treatment lamps 82 depends on the desired treatment. Desired treatments include, but are not limited to, skin disorders, such as acne vulgaris, atopic dermatitis, psoriasis, vitiligo, scleroderma, eczema, fine lines and wrinkles, as well as neonatal jaundice and some forms of cancer. For example, if the desired treatment is for skin acne blue and red LEDs would be used, as these frequencies are most useful for acne treatment. A minimum number of treatment lamps 82 are intended, with there still being enough to provide effective treatment.

In the course of treatment, the light energy from treatment lamps 82 may be manipulated to improve wearable phototherapy system 10 performance. Methods of manipulating the light energy from treatment lamps 82 may include continuous, pulsed, focused, diffuse, multi-wavelength, single wavelength, visible and/or non-visible light wavelengths.

As mentioned above frame 90 holds wearable lamp platform 15 in a fixed orientation such that the inner surface of the lamp platform and the inwardly facing surface of the controller unit are spaced from the user's face and the spacing between the inner surface of the lamp platform and an adjacent surface of the user's face defines a platform clearance. In some embodiments, the platform clearance is between about 5 mm and about 50 mm, preferably between about 10 mm and about 40 mm most preferably between about 12 mm and about 30 mm. The spacing between the inwardly facing surface of the controller unit and an adjacent surface of the user's face defines a controller clearance. In some embodiments, the controller clearance is between about 0 mm and about 25 mm (it is acceptable that the inwardly facing surface of the controller unit contacts the user's face). In some preferred embodiments, the controller clearance is between about 0 mm and about 20 mm, and in more preferred embodiments, the controller clearance is between about 0 mm and about 15 mm.

Rear panel 61 of controller unit 50 has inwardly facing surface 62. During use of wearable phototherapy system 10, inwardly facing surface 62 is directed toward the user's face. Charging receptacle 76 is disposed in the inwardly facing surface inwardly facing surface 62 of controller unit 50. The spacing between the charging receptacle 76 and a closest surface of the user's face defines a charging receptacle clearance.

As mentioned earlier charging receptacle 76 may be in the form of a USB receptacle. The means of recharging rechargeable power source 74 will use compatible USB cables. rechargeable power source 74. USB cables generally are made of a cord of parallel wires covered with a non-conducting sheath. Plugs are located at each end of the cord. The plugs have prongs which are partially exposed, and partially covered with a non-conducting overmold. The prongs of the USB cables act as the “male” part of the coupling, while the USB receptacle acts as the “female” part of the coupling. As with the USB receptacles, USB cables generally are available as standard USB

Type-A, Type-B, or Type-C, or the recently developed Mini-A, Mini-AB, Mini-B, Micro-A, Micro-AB, and Micro-B, and the cable and receptacle must match for proper coupling, and therefore charging, to occur.

To charge rechargeable power source 74 of controller unit 50, charging aperture cover flap 68 is displace (by rotating on a pivot line) to a position to allow a connection between “male” part of the coupling (the prongs of the USB cable) and the “female” part of the coupling (the USB receptacle disposed in charging receptacle 76).

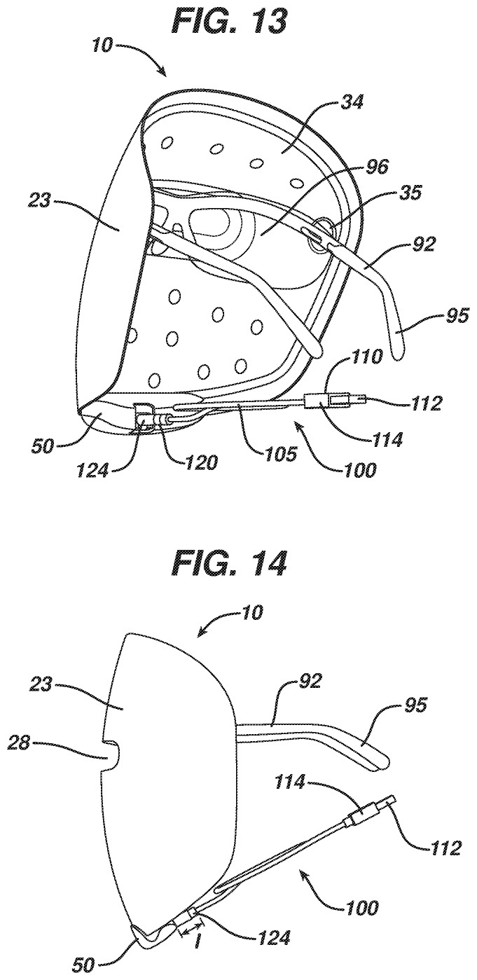

FIGS. 13 and 14 are rear and side perspective views, respectively, of phototherapy system 10 coupled to a charging cable 100. Charging cable 100 is shown with cord 105, and plugs 110 and 120. Plug 110 is shown with prong 112 and overmold 114. Overmold 124 of plug 120 is shown, with prong of plug 120 not shown, as it is inserted into charging receptacle 76. In the embodiment shown, charging cable 100 is a USB cable, plug 110 is a USB Type-A, while plug 120 is a USB Micro-A. Though shown as USB components, these components are not to meant as limiting components of the present invention.

As mentioned earlier, phototherapy system 10 disclosed herein has the safety feature of preventing the charging of system 10 while treatment is being performed. As noted earlier, the spacing between charging receptacle 76 and a closest surface of the user's face defines a charging receptacle clearance. FIG. 14 defines the length of overmold 124 of plug 120 as “1”. If the length of overmold 124 of plug 120 is greater than the charging receptacle clearance, the user will not be able to couple phototherapy system 10 to charging cable 100 while treatment is being performed. This is because the additional space required to fit the extra length of overmold 124 between the charging receptacle 76 and the closest surface of the user's face will bias phototherapy system 10 away from the user's face, and frame 90, which holds wearable lamp platform 15 in a fixed orientation for user comfort will not fit correctly onto user's face.

As mentioned above, in the embodiment shown, charging cable 100 is a USB cable. Commercially available USB cables typically have “1” values of from about 5 mm to over about 50 mm. To utilize the safety feature of the phototherapy system 10 described herein, charging receptacle clearance must be less than these “1” values. So, in some embodiments, the charging receptacle clearance is less than about 5 mm, or less than about 10 mm, or less than about 20 mm, or less than about 50 mm.

A method of treatment using the phototherapy system of claim 1 comprising inserting charging cable 100 into charging receptacle 76 for a time sufficient to energize rechargeable power source 74, then removing charging cable 100 from charging receptacle 76 to enable the user to place phototherapy system 10 on the face. In normal use, treatment lamps 82 are energized by the controller unit 50 and a program of phototherapy treatment is initiated. The program of phototherapy is managed by the controller unit 50, irradiating at least a portion of the user's face with one or more of the treatment lamps 82. When the program of phototherapy is completed, phototherapy system 10 is removed from the face.

The length of the treatment cycle will depend on treatment being performed. In some embodiments, treatment cycle is less than sixty (60) minutes, or thirty (30) minutes, or ten (10) minutes, or five (5) minutes, or one (1) minute.

The present invention will be further understood by reference to the following specific Examples which are illustrative of the composition, form and method of producing the present invention. It is to be understood that many variations of composition, form and method of producing this would be apparent to those skilled in the art. The following Examples, wherein parts and percentages are by weight unless otherwise indicated, are only illustrative.

### EXAMPLES

### Example 1

### Prototype Phototherapy Systems

Prototype phototherapy systems were constructed by converting a commercially available light therapy mask. Specifically, commercially available NEUTROGENA LIGHT THERAPY ACNE MASKS (Johnson & Johnson, New Brunswick, N.J.). For each mask, the existing cords and controllers were removed and new modules that contained the electronics were installed to the base products.

For each prototype, the new module consisted of a printed circuit board and battery. The module was installed to the two part housing of the existing mask, and the output of the circuit board was connected to the LED string leads in the mask as the final electrical connection. This module subassembly was then glued to the base of the mask utilizing cyanoacrylate adhesive. Once complete the rechargeable battery was charged through the exposed micro USB port on the inside of the module and charged for 6 hours.

To demonstrate function, once each mask was charged, the mask was turned on utilizing an on/off button located on the front of the module. Once turned on the program in the microcontroller in the module kept the mask illuminated for a preprogrammed 10-minute use time and regulated the power supplied to the LED's such that they delivered consistent optical power over the use time.

Each prototype demonstrated full functionality of the phototherapy system in the format as described in the specification.

What is claimed is:

1. A phototherapy system wearable on a portion of a user's face, the system comprising:
(a) a lamp platform having an outer surface and an opposite, inner surface directed toward the user's face during use and comprising a plurality of treatment lamps arranged and configured to irradiate a portion of the user's face;
(b) a controller unit disposed on, and electrically coupled to, the lamp platform, the controller unit having:
(i) an inwardly facing surface directed toward the user's face during use;
(ii) a rechargeable power source; and
(iii) a charging receptacle disposed in the inwardly facing surface and in electrical communication with the charging receptacle;
(c) a frame for holding the lamp platform in a fixed orientation such that the inner surface of the lamp platform and the inwardly facing surface of the controller unit are spaced from the user's face and the spacing between the inner surface of the lamp platform and an adjacent surface of the user's face defines a platform clearance and the spacing between the charging receptacle and a closest surface of the user's face defines a charging receptacle clearance.

2. The phototherapy system of claim 1 wherein the platform clearance is between about 5 mm and about 50 mm.

3. The phototherapy system of claim 1 wherein the charging receptacle clearance is less than about 25 mm.

4. The phototherapy system of claim 3 wherein the charging receptacle clearance is less than about 20 mm.

5. The phototherapy system of claim 1 wherein at least one of the plurality of treatment lamps of the wearable lamp platform provides blue light having a peak wavelength of between about 450 nm and about 495 nm.

6. The phototherapy system of claim 1 wherein at least one of the plurality of treatment lamps of the wearable lamp platform provides red light having a peak wavelength of between about 620 nm and about 700 nm.

7. The phototherapy system of claim 1 wherein at least one of the plurality of treatment lamps of the wearable lamp platform provides infrared light having a peak wavelength of between about 700 nm and about 1000 nm.

8. A phototherapy system wearable on a portion of a user's face, the system comprising:
(a) a lamp platform having
(i) an outer surface and an opposite, inner surface directed toward the user's face during use and comprising a plurality of treatment lamps arranged and configured to irradiate a portion of the user's face;
(ii) the lamp platform has a height (h) extending from a base to a top that defines a longitudinal axis, and the lamp platform has a generally concave form in a lateral plane (perpendicular to the longitudinal axis);
(b) a controller unit disposed on and electrically coupled to the base of the lamp platform, the controller unit having an inwardly facing surface directed toward the user's face during use and a rechargeable power source, and the inwardly facing surface of the controller unit has a generally concave form in a lateral plane (perpendicular to the longitudinal axis); and
(c) a frame for holding the lamp platform in a fixed orientation such that the inner surface of the lamp platform is spaced from the user's face, and the spacing between the inner surface of the lamp platform and an adjacent surface of the user's face defines a platform clearance.

9. The phototherapy system of claim 8 wherein the rechargeable power source comprises at least two elongate, cylindrical cells defining an oblique angle therebetween in the lateral plane.

10. The phototherapy system of claim 8 wherein the controller unit further includes a charging receptacle in electrical communication with the rechargeable power source.

11. The phototherapy system of claim 10 wherein the charging receptacle is disposed in the inwardly facing surface.

12. The phototherapy system of claim 11 wherein the frame holds the inwardly facing surface of the controller unit spaced from the user's face and the spacing between the charging receptacle and a closest surface of the user's face defines a charging receptacle clearance.

13. The phototherapy system of claim 8 wherein the platform clearance is between about 5 mm and about 50 mm.

14. The phototherapy system of claim 12 wherein the charging receptacle clearance is less than about 25 mm.

15. The phototherapy system of claim 14 wherein the charging receptacle clearance is less than about 20 mm.

16. The phototherapy system of claim 8 wherein at least one of the plurality of treatment lamps of the wearable lamp platform provides blue light having a wavelength of between about 450 nm and about 495 nm.

17. The phototherapy system of claim 8 wherein at least one of the plurality of treatment lamps of the wearable lamp platform provides red light having a wavelength of between about 620 nm and about 700 nm.

18. The phototherapy system of claim 8 wherein at least one of the plurality of treatment lamps of the wearable lamp platform provides infrared light having a wavelength of between about 700 nm and about 1000 nm.

19. A method of treatment using the phototherapy system of claim 1 comprising inserting a cable connector into the charging receptacle for a time sufficient to energize the rechargeable power source, removing the cable connector from the charging receptacle to enable the user to place the phototherapy system on the face, initiating a program of phototherapy managed by the controller unit that irradiates at least a portion of the user's face with one or more of the treatment lamps, allowing the program of phototherapy to complete the phototherapy, and removing the phototherapy system from the face.
